(19) United States
(12) Patent Application Publication
(10) Pub. No.: US 2006/0159751 A1
(43) Pub. Date: Jul. 20, 2006

(54) Controlled release pharmaceutical compositions of carbidopa and levodopa

(76) Inventors: Mona Gogia, New Delhi (IN); Rajeev Shankar Mathur, Delhi (IN); Sanjeev Sethi, Faridabad (IN)

Correspondence Address: RANBAXY INC., 600 COLLEGE ROAD EAST, SUITE 2100, PRINCETON, NJ, 08540, US

(21) Appl. No.: 10/510,468

(22) PCT Filed: Apr. 11, 2003
(86) PCT No.: PCT/IB03/01361

§ 371 Date: Mar. 21, 2006

(30) Foreign Application Priority Data
Apr. 11, 2002 (IN) 447/DEL/2002

(51) Int. Cl. A61K 31/198; A61K 31/717; A61K 9/22

(52) U.S. Cl. 424/468; 514/57; 514/567

(57) ABSTRACT

The present invention relates to controlled release pharmaceutical compositions of carbidopa and levodopa that include a combination of different molecular weight cellulose ethers and in particular, hydroxypropyl cellulose ether.

### FIELD OF THE INVENTION

The present invention relates to controlled release pharmaceutical compositions of carbidopa and levodopa that include a combination of different molecular weight cellulose ethers and in particular, hydroxypropyl cellulose ether.

### BACKGROUND OF THE INVENTION

Controlled drug delivery includes both sustained and extended delivery and targeted delivery on a one time or sustained basis. Controlled release formulations can be used to reduce the amount of drug necessary to cause the same therapeutic effect in patients. The convenience of fewer, but more effective doses, also increases patient compliance.

Parkinson's disease is associated with the depletion of dopamine from cells in the corpus striatum. Dopamine does not cross the blood-brain barrier and cannot, therefore, be delivered in that form to treat Parkinson's disease. Its immediate precursor, levodopa, is administered instead because it penetrates into the brain through the blood-brain barrier where it is decarboxylated to dopamine. Levodopa, however, also is decarboxylated to dopamine in peripheral tissues and consequently only a small portion of administered levodopa is transported unchanged to the brain. The decarboxylation reaction can be blocked by carbidopa, which inhibits decarboxylation of peripheral levodopa but cannot itself cross the blood-brain barrier and hence has no effect on the metabolism of levodopa in the brain.

The combination of carbidopa and levodopa is considered to be the most effective treatment for symptoms of Parkinson's disease. However, after taking carbidopa/levodopa immediate-release formulations for several years, some patients find that the effect of the medication begins to wear off well before the scheduled time for administration of the next dose. Various responses to this problem have been proposed, e.g., shorten the intervals between immediate-release doses (or add an additional dose if needed); switch from immediate-release to controlled release carbidopa/levodopa formulations.

Because of the disadvantages of increasing dose frequency and amount, leading to reduced patient compliance, a number of research endeavors have been directed towards preparing controlled release formulations of carbidopa and levodopa.

For example, U.S. Pat. No. 4,424,235 discloses hydrodynamically balanced controlled release formulations containing both L-Dopa and a decarboxylase inhibitor. The capsules and tablets are hydrodynamically balanced to have a bulk density (specific gravity) of less than 1 and, therefore, remain floating in gastric fluid, which has a specific gravity of between 1.004 and 1.010. The controlled release formulations are described as including a mixture of the active ingredients with a single polymer selected from a natural gum, methylcellulose, hydroxypropyl methylcellulose, hydroxypropyl cellulose and sodium carboxymethyl cellulose. The formulations further contain fatty materials to make the drug float in the stomach, where the polymer vehicle releases the drug. The dosage form remains buoyant and freely floating in the gastric fluid for an extended period of time during which almost the entire medicament contained in the formulation is released into the gastric fluid. A disadvantage of the floating system, however, is that it must remain buoyant even while absorbing gastric fluid.

PCT application WO 02/00213 discloses the use of non-hydrated hydrogel, super disintegrant and tannic acid to provide a gastroretentive dosage form of levodopa. The dosage form expands upon contact with gastric fluid to promote its retention in the patient's stomach for a prolonged period of time and thereby provide sustained release of the drug.

The retention of the drug in a tablet or other dosage form beyond the duration of the fed mode raises a number of problems that detract from the therapeutic efficacy of the drug. These problems arise from the tendency of the tablet to pass from the stomach into the small intestine and reach the colon with the drug still in the tablet. This is especially problematic when the patient is no longer in the fed mode. This loss of effectiveness is problematic for those drugs that provide their maximum benefit with minimum side effects when absorbed in the stomach and upper gastrointestinal tract rather than the colon. The reasons for this site specific effectiveness are either favorable condition in the stomach, unfavorable conditions in the colon, or both.

To overcome the disadvantages of a gastroretentive dosage form, controlled release dosage forms of carbidopa and levodopa have been prepared by embedding the active ingredient into a polymer matrix that slowly erodes to release the active. U.S. Pat. Nos. 4,832,957 and 4,900,755 describe controlled release formulations of levodopa/carbidopa in which the desired controlled release is achieved by using a polymer vehicle that includes a combination of water-soluble and less soluble polymers.

The water soluble polymers in these patents are described as being selected from hydroxypropyl cellulose, hydroxypropyl methylcellulose, polyvinyl pyrrolidone, polyethylene glycol, starch, and methyl cellulose. The less water-soluble polymers are selected from polyvinyl acetate-crotonic acid copolymer, polyvinyl chloride, polyethylene, cellulose acetate, polyvinyl alcohol, ethylene vinyl acetate copolymer, polyvinyl acetate, polymethyl methacrylate, ethyl cellulose, and the like. According to these patents, the preferred polymeric vehicle is disclosed as being a combination of water-soluble polymer, hydroxypropyl cellulose, and the less water soluble polymer polyvinyl acetate-crotonic acid.

Although these patents describe the use of a combination of water soluble and less water-soluble polymers for preparing a control release formulation of carbidopa and levodopa, they do not suggest the use of a combination of different molecular weights of a single cellulose ether.

U.S. Pat. No. 4,389,393 discloses a formulation for the controlled release of a medicament by using a polymer vehicle that is a combination of hydroxypropyl cellulose and hydroxypropyl methylcellulose. However, this patent does not suggest the combination of different molecular weights of a single cellulose ether.

U.S. Pat. No. 6,103,263 describes the use of two types of hydroxypropyl cellulose, one of which has a low molecular weight and the other of which has a high molecular weight. The two hydroxypropyl celluloses are used to obtain a pharmaceutical formulation having delayed-pulse, sustained release characteristics over at least 12 hour period. The low molecular weight hydroxypropyl cellulose ethers are disclosed as having a number average molecular weight of 70,000 to 90,000 and the high molecular weight hydroxypropyl cellulose is disclosed as having a number average molecular weight of 1,100,000 to 1,200,000.

A key disclosure in U.S. Pat. No. 6,103,263 is that the desired sustained release characteristics of the active ingredient can be ensured by a ratio of between 1:1.6 to 1:8.3, and preferably 1:4, of the active ingredient to the mixture of low molecular weight hydroxypropyl cellulose and high molecular weight hydroxypropyl cellulose. It further describes the ratio of low molecular weight hydroxypropyl cellulose to high molecular weight hydroxypropyl cellulose as being from 1:2 to 1:15. The total polymer content amounts to between 24 to 70% by weight of the composition.

A disadvantage of this formulation is the increased cost that results from the higher polymer concentration. Moreover, U.S. Pat. No. 6,103,263 does not suggest the use of a combination of low molecular weight hydroxypropyl cellulose and medium molecular weight hydroxypropyl cellulose.

### SUMMARY OF THE INVENTION

In one general aspect, there is provided a controlled release pharmaceutical composition that includes carbidopa, levodopa, and a combination of a low molecular weight cellulose ether and a medium molecular weight cellulose ether. The low molecular weight cellulose ether and the medium molecular weight cellulose ether are the same type of cellulose ether.

Embodiments of the pharmaceutical composition may include one or more of the following features. For example, the first and the second cellulose ethers may be hydroxypropyl cellulose ethers or hydroxypropyl methyl cellulose ethers.

The low molecular weight cellulose ether may be hydroxypropyl cellulose ether having a number average molecular weight of between approximately 55,000 and approximately 70,000 and, more particularly, approximately 65,000. The medium molecular weight cellulose ether may be hydroxypropyl cellulose ether having a number average molecular weight of between approximately 110,000 and approximately 150,000 and, more particularly, approximately 125,000.

The ratio of low molecular weight cellulose ether to medium molecular weight cellulose ether may be approximately 0.75:1 to 1.5:1 and, more particularly, approximately 1:1. The total cellulose ether concentration may be between approximately 2% and approximately 20% w/w of the composition.

The controlled release pharmaceutical composition may further include one or more pharmaceutical excipients. The one or more pharmaceutical excipients may be one or more diluents, binders, disintegrants, lubricants, glidants, colorants, and flavoring agents. The controlled release pharmaceutical composition may be a tablet.

In another general aspect, there is provided a process for the preparation of a controlled release composition of carbidopa and levodopa. The process includes blending carbidopa, levodopa, a low molecular weight cellulose ether, and a medium molecular weight cellulose ether, optionally granulating the blend with a binder, and compressing into a tablet. The first cellulose ether and the second cellulose ether are the same type of cellulose ether.

Embodiments of the process may include one or more of the following features. For example, the low molecular weight cellulose ether and the medium molecular weight cellulose ether may be hydroxypropyl cellulose ether or hydroxypropyl methyl cellulose ether.

The low molecular weight cellulose ether may be hydroxypropyl cellulose ether having a number average molecular weight of between approximately 55,000 and approximately 70,000. The medium molecular weight cellulose ether may be hydroxypropyl cellulose ether having a number average molecular weight of approximately 110,000 to approximately 150,000.

The granules may be prepared by either of a wet granulation or a dry granulation technique. The wet granulation may be performed with one or more of an aqueous, hydro-alcoholic, or alcoholic dispersion of the binder.

In another general aspect, there is provided a method of providing dopamine to the brain. The method includes administering a tablet that includes carbidopa, levodopa, a low molecular weight cellulose ether, and a medium molecular weight cellulose ether.

Embodiments of the method may include one or more of the following features. For example, the low molecular weight cellulose ether may be hydroxypropyl cellulose ether having a number average molecular weight of between approximately 55,000 and approximately 70,000. The medium molecular weight cellulose ether may be hydroxypropyl cellulose ether having a number average molecular weight of between approximately 110,000 and approximately 150,000.

In another general aspect, a method of treating Parkinson's disease includes administering a pharmaceutical composition to a patient in need of treatment for Parkinson's disease. The pharmaceutical composition administered includes carbidopa, levodopa, a low molecular weight cellulose ether, and a medium molecular weight cellulose ether. The low molecular weight cellulose ether and the medium molecular weight cellulose ether are the same type of cellulose ether.

The low molecular weight cellulose ether may be hydroxypropyl cellulose ether having a number average molecular weight of between approximately 55,000 and approximately 70,000. The medium molecular weight cellulose ether may be hydroxypropyl cellulose ether having a number average molecular weight of between approximately 110,000 and approximately 150,000.

### DETAILED DESCRIPTION OF INVENTION

The inventors have discovered two important characteristics in developing a controlled release formulation of carbidopa and levodopa: (1) the formulation can be prepared using a combination of low and medium molecular weight cellulose ether polymers, such as hydroxypropyl cellulose ethers, and (2) the cellulose ether polymers can be provided in a low concentration and yet the formulation produces the desired release profile. The resulting tablet or other dosage form maintains relatively steady plasma levodopa levels for four to six hours. The inventors further found that the use of either low molecular weight or medium molecular weight hydroxypropyl cellulose without the other did not give the desired dissolution profile.

The compositions produced by the present process are quite stable and provide comparable dissolution release profiles when compared to Bristol Myers Squibb's Sinemet® CR (the commercially marketed carbidopa/levodopa controlled release tablets). Because the present process employs low concentration of the polymer, the cost of the production is considerably reduced.

As noted above, the compositions include carbidopa, levodopa, at least two cellulose ethers that are of the same type but one is of a low molecular weight and the other is of a medium molecular weight, and one or more pharmaceutically acceptable excipients or additives. The carbidopa may be present in the composition at between approximately 5 mg and 300 mg and levodopa may be present at an amount that is between approximately 20 mg and 1200 mg.

The cellulose ethers may be selected from either low and medium molecular weight hydroxypropyl cellulose ether or hydroxypropyl methyl cellulose ether. The low molecular weight hydroxypropyl cellulose may be selected from hydroxypropyl cellulose having a number average molecular weight of between approximately 55,000 and approximately 70,000 and the medium molecular weight hydroxypropyl cellulose may be selected from hydroxypropyl cellulose having a number average molecular weight of between approximately 110,000 to 150,000. However, the combination of hydroxypropyl cellulose ether having a number average molecular weight of between about 65,000 and about 125,000 is particularly suitable for effectively delivering a combination of carbidopa and levodopa.

The concentration of the combination of the low and the medium molecular weight hydroxypropyl cellulose ethers may vary and be as much as 20% or as little as 2% by weight of the total composition. For example, as shown in the examples, it was discovered that one suitable range is between 5% and 16% w/w. The ratio of low molecular weight hydroxypropyl cellulose to medium molecular weight hydroxypropyl cellulose may vary from approximately 0.75:1 to approximately 1.5:1. However, a ratio of about 1:1 is preferred.

In addition to the active ingredients and the cellulose ether, the formulation may include pharmaceutically acceptable additives or excipients, which act in one or more capacities as diluents, binders, disintegrants, lubricants, glidants, colorants or flavoring agents. For example, diluents may be selected from lactose, mannitol, sucrose, microcrystalline cellulose, starches, calcium hydrogen phosphate, and other suitable, known diluents. Disintegrants may be selected from croscarmellose sodium, crospovidone, sodium starch glycolate, and other suitable, known disintegrants.

Binders impart cohesiveness to the blend and also improve the flow and hardness. Binders may be selected from excipients, such as starch, sugars, gums, povidone, and other suitable, known binders.

Lubricants may be selected from talc, magnesium stearate, calcium stearate, polyethylene glycol, hydrogenated vegetable oils, stearic acid, sodium lauryl sulphate, sodium stearyl fumarate, sodium benzoate, and other suitable, known lubricants. Glidants may be selected from colloidal silicon dioxide, aerosol, talc, and other suitable, known glidants. Suitable coloring and flavoring agents include those approved for use by the United States Food and Drug Administration (FDA) and are well known to those skilled in the art.

The formulation may be prepared by dry blending levodopa and carbidopa with a combination of low molecular weight hydroxypropyl cellulose and medium molecular weight hydroxypropyl cellulose, wet granulating the blend with an aqueous solution of binder, drying and sizing the wet granules, and compressing the granules. Although wet granulation works very well in forming the dosage forms, direct compression and dry granulation techniques may instead be used to prepare tablets. The tablets can be optionally coated using any standard coating process.

The following examples are provided to enable one of ordinary skill in the art to prepare dosage forms of the invention and should not be construed as limiting the scope of the invention. In the following examples, the carbidopa/levodopa tablets were prepared using the polymer being present at between approximately 2% and approximately 20% w/w of the total composition.

### EXAMPLES 1-8

| | Weight (mg per tablet) | | | | | | | |
|---|---|---|---|---|---|---|---|---|
| Ingredient | 1 | 2 | 3 | 4 | 5 | 6 | 7 | 8 |
| Carbidopa | 54.91 | 59.28 | 54.91 | 54.91 | 54.91 | 54.91 | 54.39 | 54.94 |
| Levodopa | 201.35 | 201.35 | 201.35 | 201.35 | 201.35 | 201.35 | 201.73 | 201.73 |
| Microcrystalline | 50.515 | 26.35 | 5.04 | 8.04 | 10.04 | 20.04 | 12.88 | 9.64 |
| cellulose | | | | | | | | |
| HPC-L* | 15.0 | 25.0 | 15 | 15 | 12.5 | 7.5 | 12.5 | 12.5 |
| HPC-M** | 20.0 | 30.0 | 15 | 12 | 12.5 | 7.5 | 10 | 12.5 |
| Povidone K-30 | 3.5 | 3.5 | 3 | 3 | 3 | 3 | 3 | 3 |
| Iron oxide red | 0.35 | 0.35 | 0.2 | 0.2 | 0.2 | 0.2 | 0.25 | 0.3 |
| D & C yellow | 0.875 | 0.875 | 0.5 | 0.5 | 0.5 | 0.5 | 0.25 | 0.4 |
| no. 10 | | | | | | | | |
| Granulating | q.s. | q.s. | q.s | q.s | q.s | q.s | q.s | q.s |
| fluid*** | | | | | | | | |
| Magnesium | 3.5 | 3.5 | 5 | 5 | 5 | 5 | 5 | 5 |
| stearate | | | | | | | | |

*HPC-L = Low molecular weight hydroxypropyl cellulose,
**HPC-M = Medium molecular weight hydroxypropyl cellulose,
***Granulating fluid = Water, alcohol or mixture of both

Process:
1. Each of the ingredients was sieved to the appropriate size and the required amount of each ingredient was weighed out.
2. A solution of povidone was prepared in granulating fluid.
3. Carbidopa, levodopa, hydroxypropyl cellulose, microcrystalline cellulose, and colorants were blended.
4. The blend of step 3 was granulated using the povidone solution of step 2 and the resulting granules were dried and sized.
5. The sized granules of step 4 were lubricated with magnesium stearate and compressed into suitable sized tablets.

Table 1 provides comparative dissolution data for the marketed Sinemet® CR and the controlled release tablets of carbidopa/levodopa of examples 1-8. The testing was performed in 0.1N HCl (900 ml), USP 2 at 50 rpm. As indicated below in Table 1, the controlled release tablets prepared according to the above examples provide a sustained release of carbidopa and levodopa for at least 2.5 hours. The dissolution profile clearly shows that a 0.75:1-1.5:1 ratio of low to medium molecular weight hydroxypropyl cellulose provides an extended release profile that is similar to that of Sinemet® CR. Moreover, the desired release of carbidopa and levodopa can be achieved using a low concentration of hydroxypropyl cellulose.

TABLE 1
Comparative dissolution of the controlled release tablets of Carbidopa levodopa prepared as per Examples 1-8 and
Sinemet ® CR in 0.1N HCl (900 ml), USP 2 at 50 rpm.

| | % drug released | | | | | | | | | | | | | | | | | |
|---|---|---|---|---|---|---|---|---|---|---|---|---|---|---|---|---|---|---|
| | | | | | | | | | | | | | | | | | Sinemet ® | |
| Time | Ex. 1 | | Ex. 2 | | Ex. 3 | | Ex. 4 | | Ex. 5 | | Ex. 6 | | Ex. 7 | | Ex. 8 | | CR | |
| (h) | C | L | C | L | C | L | C | L | C | L | C | L | C | L | C | L | C | L |
| 0.5 | 36 | 35 | 27 | 26 | 36 | 38 | 37 | 38 | 31 | 31 | 29 | 34 | 33 | 35 | 31 | 34 | 33 | 35 |
| 1.0 | 68 | 66 | 50 | 48 | 54 | 57 | 60 | 63 | 53 | 52 | 54 | 55 | 65 | 69 | 57 | 59 | 59 | 61 |
| 1.5 | 89 | 87 | 74 | 70 | 75 | 79 | 78 | 81 | — | — | — | — | — | — | — | — | — | — |
| 2.0 | 101 | 99 | 91 | 86 | 87 | 92 | 87 | 92 | — | — | — | — | — | — | — | — | — | — |
| 2.5 | 107 | 106 | 104 | 97 | 98 | 103 | 95 | 100 | 98 | 96 | 97 | 95 | 103 | 108 | 93 | 96 | 97 | 98 |

C—Carbidopa
L—Levodopa

### EXAMPLES 9-10

Weight (mg per tablet)

| Ingredient | 9 | 10 |
|---|---|---|
| Carbidopa | 25.0 | 50.0 |
| Levodopa | 100.0 | 200.0 |
| Microcrystalline cellulose | 6.226 | 12.452 |
| HPC-L* | 6.25 | 12.5 |
| HPC-M** | 6.25 | 12.5 |
| Povidone K-30 | 1.5 | 3.0 |
| Iron oxide red | 0.1250 | 0.25 |
| Iron oxide yellow | 0.1250 | 0.25 |
| Granulating fluid*** | q.s. | q.s. |
| Magnesium stearate | 2.5 | 5.0 |

*HPC-L = Low molecular weight hydroxypropyl cellulose,
**HPC-M = Medium molecular weight hydroxypropyl cellulose,
***Granulating fluid = Water, alcohol or mixture of both

Process:
1. Each of the ingredients was sieved to the appropriate size and the required amount of each ingredient was weighed out.
2. A solution of povidone was prepared in granulating fluid.
3. Carbidopa, levodopa, hydroxypropyl cellulose, microcrystalline cellulose and part of the colorants were blended.
4. The blend of step 3 was granulated using the povidone solution of step 2, dried, and sized.
5. The sized granules of step 4 were blended with the remaining amount of colorants, lubricated with magnesium stearate, and compressed into suitably sized tablets.

Table 2 provides comparative dissolution data for the marketed Sinemet® CR and the controlled release tablets of carbidopa/levodopa of Examples 9 and 10. The testing was performed in 0.1N HCl (900 ml), USP 2 at 50 rpm. The controlled release tablets prepared according to Examples 9 and 10 provide controlled release of carbidopa and levodopa for 4 hours. The dissolution profile clearly shows that a 0.75:1-1.5:1 ratio of low to medium molecular weight hydroxypropyl cellulose provides an extended release profile that is similar to that of Sinemet® CR. Moreover, the desired release of carbidopa and levodopa can be achieved using a low concentration of hydroxypropyl cellulose.

TABLE 2
Comparative dissolution of the controlled release tablets of
carbidopa and levodopa prepared as per Examples 9-10 and
Sinemet ® CR in 0.1N HCl (900 ml), USP 2 at 50 rpm.

| | % drug released | | | | | | | |
|---|---|---|---|---|---|---|---|---|
| | | | | | Sinemet ® CR | | | |
| | Ex. 9 | | Ex. 10 | | Batch 1 | | Batch 2 | |
| Time (h) | C | L | C | L | C | L | C | L |
| 0.5 | 36 | 38 | 29 | 29 | 36 | 39 | 36 | 36 |
| 1.0 | 59 | 61 | 50 | 50 | 57 | 59 | 58 | 59 |
| 2.5 | 96 | 99 | 89 | 89 | 92 | 95 | 97 | 100 |
| 4.0 | 100 | 103 | 104 | 105 | 97 | 99 | 100 | 104 |

C—Carbidopa
L—Levodopa

The compositions and tablets described above can be administered to patients in need of increased dopamine levels in the brain e.g., patients suffering from Parkinson's disease. By administering the composition in the form of tablets or other dosage form, the patient is ultimately provided with dopamine to the brain.

While several particular forms of the invention have been illustrated and described, it will be apparent that various modifications and combinations of the invention detailed in the text can be made without departing from the spirit and scope of the invention. For example, a low molecular weight and a medium molecular weight hydroxypropyl methyl cellulose ether can be used in the composition in place of the hydroxypropyl cellulose ethers. Further, it is contemplated that any single feature or any combination of optional features of the inventive variations described herein may be specifically excluded from the claimed invention and be so described as a negative limitation. Accordingly, it is not intended that the invention be limited, except as by the appended claims.

We claim:

1. A controlled release pharmaceutical composition, the composition comprising carbidopa, levodopa, and a combination of a low molecular weight cellulose ether and a medium molecular weight cellulose ether, wherein the low molecular weight cellulose ether and the medium molecular weight cellulose ether are the same type of cellulose ether.

2. The controlled release pharmaceutical composition of claim 1, wherein the low molecular weight cellulose ether and the medium molecular weight cellulose ether comprise hydroxypropyl cellulose ether.

3. The controlled release pharmaceutical composition of claim 1, wherein the low molecular weight cellulose ether and the medium molecular weight cellulose ether comprise hydroxypropyl methyl cellulose ether.

4. The controlled release pharmaceutical composition of claim 1, wherein the low molecular weight cellulose ether comprises hydroxypropyl cellulose ether having a number average molecular weight of between approximately 55,000 and approximately 70,000.

5. The controlled release pharmaceutical composition of claim 4, wherein the low molecular weight hydroxypropyl cellulose has a number average molecular weight of approximately 65,000.

6. The controlled release pharmaceutical composition of claim 1, wherein the medium molecular weight cellulose ether comprises hydroxypropyl cellulose ether having a number average molecular weight of between approximately 110,000 and approximately 150,000.

7. The controlled release pharmaceutical composition of claim 6, wherein the medium molecular weight hydroxypropyl cellulose has a number average molecular weight of approximately 125,000.

8. The controlled release pharmaceutical composition of claim 1, wherein a ratio of low molecular weight cellulose ether to medium molecular weight cellulose ether is approximately 0.75:1 to 1.5:1.

9. The controlled release pharmaceutical composition of claim 1, wherein a ratio of low molecular weight cellulose ether to medium molecular weight cellulose ether is approximately 1:1.

10. The controlled release pharmaceutical composition of claim 1, wherein the total cellulose ether concentration is between approximately 2% and approximately 20% w/w of the composition.

11. The controlled release pharmaceutical composition of claim 1, wherein the pharmaceutical composition comprises a tablet.

12. The controlled release pharmaceutical composition of claim 1, further comprising one or more pharmaceutical excipients.

13. The controlled release pharmaceutical composition of claim 12, wherein the one or more pharmaceutical excipients comprise one or more diluents, binders, disintegrants, lubricants, glidants, colorants, and flavoring agents.

14. A process for the preparation of a controlled release composition of carbidopa and levodopa, the process comprising: blending carbidopa, levodopa, a low molecular weight cellulose ether, and a medium molecular weight cellulose ether; optionally granulating the blend with a binder; and compressing into a tablet, wherein the low molecular weight cellulose ether and the medium molecular weight cellulose ether are the same type of cellulose ether.

15. The process of claim 14, further comprising blending with one or more pharmaceutically acceptable excipients.

16. The process of claim 14, wherein the low molecular weight cellulose ether and the medium molecular weight cellulose ether comprise hydroxypropyl cellulose ether.

17. The process of claim 14, wherein the low molecular weight cellulose ether comprises hydroxypropyl cellulose ether having a number average molecular weight of between approximately 55,000 and approximately 70,000.

18. The process of claim 14, wherein the medium molecular weight cellulose ether comprises hydroxypropyl cellulose ether having a number average molecular weight of approximately 110,000 to approximately 150,000.

19. The process of claim 14, wherein granulating comprises one of a wet granulation or a dry granulation technique.

20. The process of claim 19, wherein the wet granulation is done with one or more of an aqueous, hydro-alcoholic, or alcoholic dispersion of the binder.

21. A method of providing dopamine to the brain, the method comprising administering a tablet comprising carbidopa, levodopa, a low molecular weight cellulose ether, and a medium molecular weight cellulose ether, wherein the low molecular weight cellulose ether and the medium molecular weight cellulose ether are the same type of a cellulose ether.

22. The method of claim 21, wherein the low molecular weight cellulose ether comprises hydroxypropyl cellulose ether having a number average molecular weight of between approximately 55,000 and approximately 70,000.

23. The method of claim 21, wherein the medium molecular weight cellulose ether comprises hydroxypropyl cellulose ether having a number average molecular weight of approximately 110,000 to approximately 150,000.

24. A method of treating Parkinson's disease, the method comprising administering a pharmaceutical composition to a patient in need of treatment for Parkinson's disease, the pharmaceutical composition comprising carbidopa, levodopa, a low molecular weight cellulose ether, and a medium molecular weight cellulose ether, wherein the low molecular weight cellulose ether and the medium molecular weight cellulose ether are the same type of cellulose ether.

25. The method of claim 24, wherein the low molecular weight cellulose ether comprises hydroxypropyl cellulose ether having a number average molecular weight of between approximately 55,000 and approximately 70,000.

26. The method of claim 24, wherein the medium molecular weight cellulose ether comprises hydroxypropyl cellulose ether having a number average molecular weight of approximately 110,000 to approximately 150,000.
